(19) United States
(12) Patent Application Publication
(10) Pub. No.: US 2026/0061163 A1
(43) Pub. Date: Mar. 5, 2026

(54) CATHETER HAVING A DISTAL END WITH A MULTIPLE LAYER REINFORCEMENT

(71) Applicant: DePuy Synthes Products, Inc., Raynham, MA (US)

(72) Inventors: Andres RUIZ, Miami, FL (US); Pedro D. PEDROSO, Parkland, FL (US)

(73) Assignee: DePuy Synthes Products, Inc., Raynham, MA (US)

(21) Appl. No.: 18/818,734

(22) Filed: Aug. 29, 2024

(51) Int. Cl. A61M 25/00

(52) U.S. Cl. CPC A61M 25/0053; A61M 2025/0059; A61M 2205/02

(57) ABSTRACT

A catheter for navigating within the vasculature of a body includes a liner, a braid disposed about the liner, and a jacket disposed about the braid. The liner has a proximal end, a distal end, an outer surface, an internal lumen extending from the proximal end to the distal end, and a longitudinal axis. The braid is disposed on the outer surface of the liner. The braid has a proximal end, and a distal end. The braid extends from the proximal end of the liner to the distal end of the liner. The distal end of the braid is folded over upon itself by a predetermined distance to form a first fold line and a double braid comprising of a radially inner braid layer and a radially outer layer at a distal segment of the braid.

### FIELD

The present disclosure generally relates devices and methods for improving the flexibility of the distal end of a catheter while maintaining the catheter's ability to resist mechanical deformations, such as, for example, crushing, stretching, or collapsing while being used in blood vessels during intravascular medical treatments.

### BACKGROUND

Catheters are used in intravascular medical treatments, such as, for example, for treating aneurysms and for mechanical thrombectomy for endovascular intervention, often in cases where patients are suffering from conditions such as acute ischemic stroke (AIS), myocardial infarction (MI), and pulmonary embolism (PE). Accessing the neurovascular bed is challenging with conventional technology, as the target vessels are small in diameter, remote relative to the site of insertion, and highly tortuous. These catheters are frequently of great length and must follow the configuration of the blood vessels in respect of all branching and windings. Traditional devices are often either too large in profile, lack the deliverability and flexibility needed to navigate particularly tortuous vessels, or are not effective at removing a clot when delivered to the target site.

Catheters typically have a reinforcement layer to resist mechanical deformations, such as, for example, crushing, stretching, collapsing, etc. Catheters typically lower the reinforcement layer closest to the distal end to maintain flexibility as the catheter traverses through tortuous anatomy. However, a reduced reinforcement layer in the distal end also reduces the catheters resistance to mechanical deformations.

As a result, there remains a need for improved catheter designs that refine current methods for improving the flexibility of the catheter to improve the trackability of the catheter. The presently disclosed designs are aimed at providing an improved catheter having improved deliverability and flexibility needed to navigate particularly tortuous vessels. The presently disclosed designs also provide a catheter having a braided metal wire reinforcing layer capable of improving visibility of the catheter under fluoroscopy, while still being flexible, including at the distal end of the catheter, to improve the trackability of the catheter as it navigates tortuous vessels.

For vascular catheters to reach their target site a catheter is made such that, at its proximal end, the catheter is designed to be relatively stiff and gradually becomes less stiff/more flexible at the catheter distal end. This is mainly achieved by using harder materials proximally (Rockwell D from 180 to 90) and softer materials distally (Rockwell <35D to Shore 72A to 30A) for the outer jacket materials. Vascular catheters are navigated within vessels, such as, for example, in the human anatomy, with the aid of fluoroscopic imaging which utilizes x-rays. However, the polymers of catheters do not attenuate x-ray very well as the catheter is not visible under fluoroscopy. Any metal components of the catheter body, for example, a braided or coiled wire reinforcement layer, are not significantly radiopaque. Stainless steel grades, such as, for example, 304, 316, 404, etc. are often used as the braid material. However, stainless steels are moderately radiopaque but not to the level clinically acceptable to behave as a marker band. For this reason, conventional catheters incorporate a highly x-ray attenuating metal marker, called a marker band, at the very distal tip of the catheter. Thus, medical practitioners rely only on physical feedback from conventional catheter designs to aide in their navigation of such devices within the vasculature.

### SUMMARY

In some examples, a catheter in accordance with the present disclosure is disclosed for treating aneurysms. The catheter can include a catheter navigating within the vasculature of a body. The catheter includes a liner, a braid, and a jacket. The liner has a proximal end, a distal end, an outer surface, an internal lumen extending from the proximal end to the distal end, and a longitudinal axis. The braid is disposed on the outer surface of the liner. The braid has a proximal end, and a distal end. The braid extends from the proximal end of the liner to the distal end of the liner and includes a plurality of wires. The distal end of the braid is folded over upon itself by a predetermined distance to form a first fold line and a double braid comprising of a radially inner braid layer and a radially outer layer at a distal segment of the braid. The jacket is disposed about the braid and extending from the proximal end of the liner to the distal end of the liner.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1 is a perspective view of a liner with parts broken away, according to aspects of the present disclosure.

FIG. 2 is a perspective view of a liner with parts broken away and a braid disposed about the liner, according to aspects of the present disclosure.

FIG. 3A is a cross-sectional view taken from lines 3-3 of FIG. 2 and looking in the direction of the arrows.

FIG. 3B is a cross-sectional view taken from lines 3-3 of FIG. 2 and looking in the direction of the arrows illustrating an alternative embodiment.

FIG. 3C is a cross-sectional view taken from lines 3-3 of FIG. 2 and looking in the direction of the arrows illustrating another alternative embodiment.

FIG. 4 is a perspective view of a liner and a braid disposed about the liner with parts broken away, according to aspects of the present disclosure.

FIG. 5A is a cross-sectional view taken from lines 5-5 of FIG. 4 and looking in the direction of the arrows.

FIG. 5B is a cross-sectional view taken from lines 5-5 of FIG. 4 and looking in the direction of the arrows illustrating an alternative embodiment.

FIG. 6 is a perspective view of a liner and a braid disposed about the liner with parts broken away, according to aspects of the present disclosure.

FIG. 7 is a cross-sectional view taken from lines 7-7 of FIG. 6 and looking in the direction of the arrows.

FIG. 8 is a perspective view of a liner and a braid disposed about the liner with parts broken away, according to aspects of the present disclosure.

FIG. 9 is a cross-sectional view taken from lines 9-9 of FIG. 8 and looking in the direction of the arrows.

FIG. 10 is a cross-sectional view taken from lines 10-10 of FIG. 9 and looking in the direction of the arrows.

FIG. 11 is a perspective view of a liner and a braid disposed about the liner with parts broken away, according to aspects of the present disclosure.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 80%”may refer to the range of values from 61% to 99%.

As used herein, the term “microcatheter” is a catheter having a diameter that is small in comparison to catheters in cardiovascular applications, i.e., 8 French or less.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

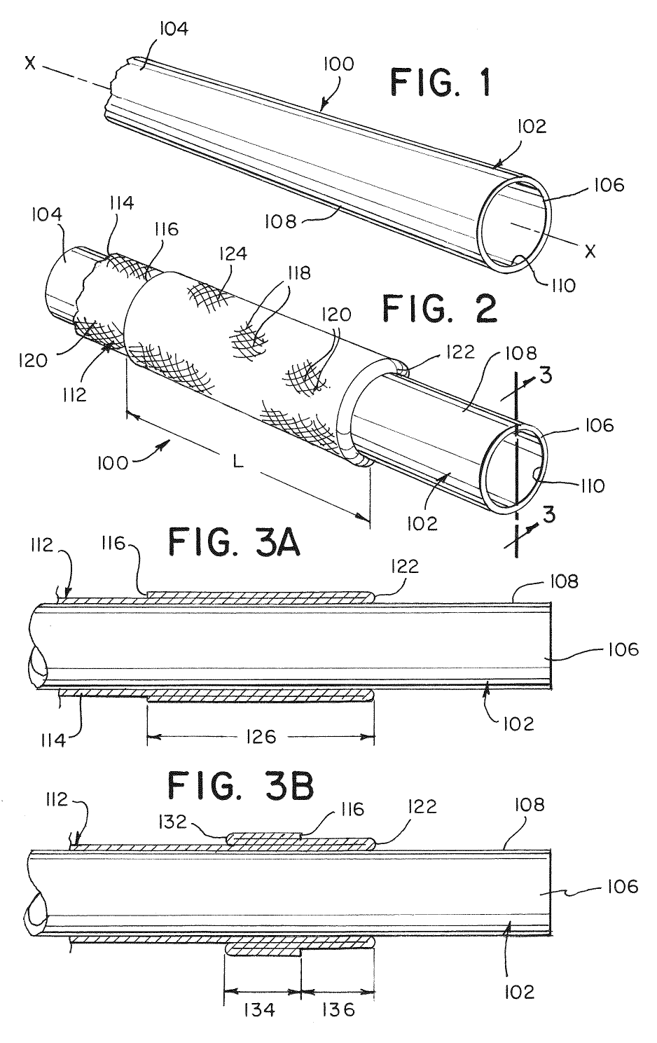

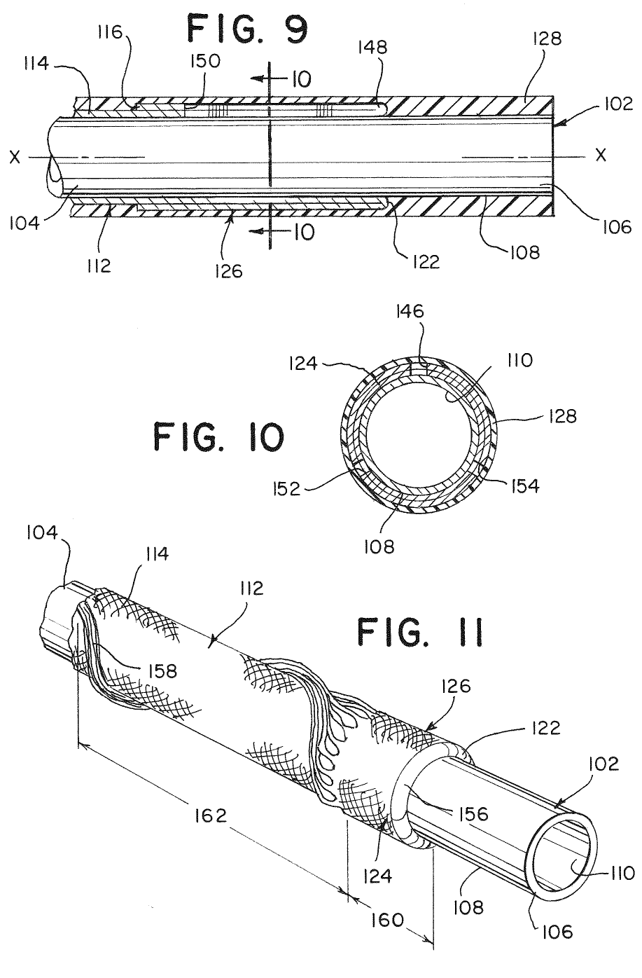

Referring now to FIGS. 1-3C, a catheter 100 for navigating within the vasculature of a body is illustrated. Catheter 100 includes a liner 102 having a proximal end 104, a distal end 106, and an outer surface 108. Liner 102 has an internal lumen 110 extending from the proximal end 104 to the distal end 106, and a longitudinal axis X. A braid 112 is disposed on the outer surface 108 of the liner 102. Braid 112 has a proximal end 114 and a distal end 116. Braid 112 extends from the proximal end 104 of the liner 102 to the distal end 106 of the liner 102. Braid 112 has a plurality of wires 118, 120. The distal end 116 of the braid is folded over upon itself by a predetermined distance L to form a first fold line 122 and a double braid section 124 comprising of a radially inner braid layer and a radially outer layer at a distal segment 126 of braid 112. A jacket 128, shown in FIG. 9, is disposed about the braid 112 and extends from the proximal end 104 of liner 102 to the distal end 106 of liner 102. The jacket 128 is disposed about the braid 112 and the liner 102 in all embodiments described herein.

Braid 112 is woven or made from wires 118, 120. Wires 118 can be made from a radiopaque material, such as, for example, tungsten or a tungsten alloy, or Platinum-Iridium or an alloy thereof. Wires 120 can be made from a different material, such as, for example, stainless steel, nitinol, a polyester or Polyethylene Terephthalate (PET), or Kevlar.

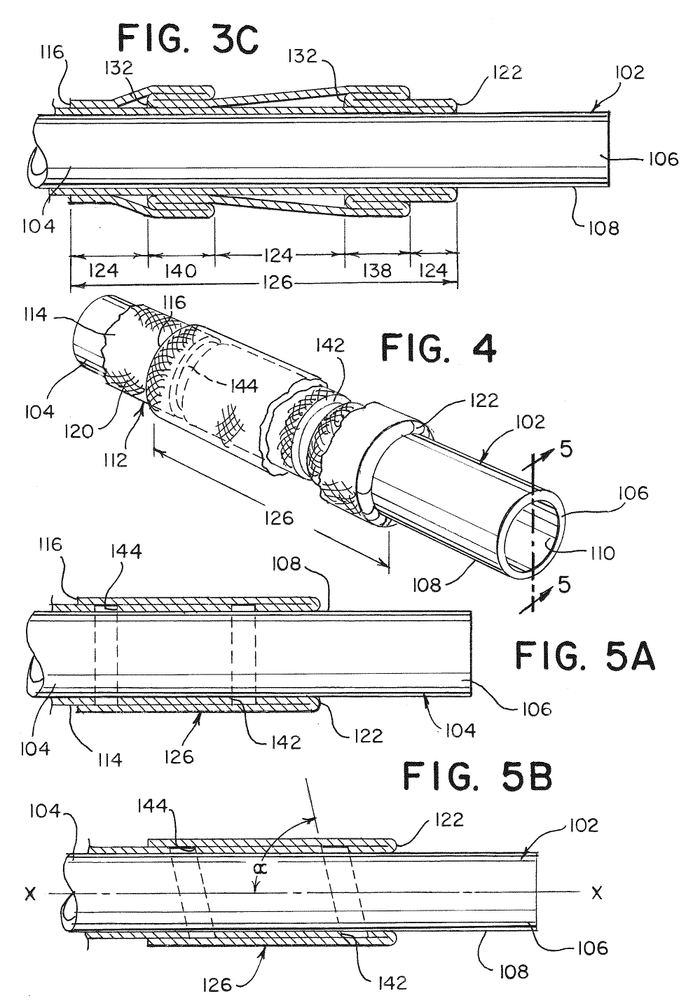

The distal end 116 of braid 112 can be folded over upon itself to form a second fold line 132 as shown in FIG. 3B. Thus, a first triple braid segment 134 can be formed for a portion of the distal segment of the braid and a double braid segment 136 can be formed for the remaining portion of the distal segment 126. The first fold line 122 is distal from the second fold line 132.

Referring now to FIG. 3C, the distal segment 126 of double braid 124 has the radially outer layer of double braid 124 being folded to create a first four-layer braid segment 138 between the distal end 116 of the braid 112 and the second fold line 132. The distal segment 126 of double braid 124 has the radially outer layer of double braid 124 being folded to create a second four-layer braid segment 140 between the distal end 116 of the braid 112 and the second fold line 132. The first four-layer braid segment 138 is distal from and spaced from the second four-layer braid segment 140.

This folding will add a dual, triple or a four-layer metal reinforcement layer increasing the catheter's radial resistance, stretch resistance, collapse resistance. If there are radiopaque wires included in the reinforcement layer, this will increase the radiopaque density of the distal end section of the catheter. By increasing the radiopaque density at the distal end of the catheter, the need for a distal end marker band is effectively removed. Thus, the distal end of the catheter can have increased flexibility while still being reinforced and visible under fluoroscopy. In an embodiment, distal segment 126 can be about 1.0 to about 7.0 cm, and approximately 5.0 cm. Also, the outer diameter of the catheter can be about 0.105 inches.

Referring now to FIGS. 4-5B, the distal segment 126 of double braid 124 has the radially inner layer of the double braid 124 having a first cut 142. First cut 142 in braid 112 is in the radially inner portion of the distal segment 126 of the braid such that the folded over portion of the distal segment of the braid is disposed radially over the first cut 126 in the braid 112. As shown in FIGS. 4 and 5A, first cut 142 in braid 112 can have an annular shape. Alternatively, as shown in FIG. 5B, first cut 142 in braid 112 can be disposed at an angle α with respect to longitudinal axis X, wherein the angle α is an acute angle. The acute angle can be from about 15 degrees to about 45 degrees.

A second cut 144 can be formed in braid 112. Like first cut 142, in the distal segment 126 of double braid 124, the radially inner layer of the double braid 124 can have a second cut 144. The second cut 144 in braid 112 is in the radially inner portion of the distal segment 126 of the braid such that the folded over portion of the distal segment 126 of the braid is disposed radially over the second cut 144 in the braid. Second cut 144 in braid 112 can have an annular shape. Alternatively, as shown in FIG. 5B, second cut 144 in braid 112 can be disposed at an angle α with respect to longitudinal axis X, wherein the angle α is an acute angle. As shown, the first cut 142 in braid 112 is axially spaced from the second cut 144 in braid 112 with first cut 142 being distal with respect to second cut 144. Cuts 142, 144 can be formed, for example, by a laser or by other suitable cutting instruments. Cuts 142, 144 will increase the flexibility of the distal end of the catheter, while still being reinforced and visible under fluoroscopy.

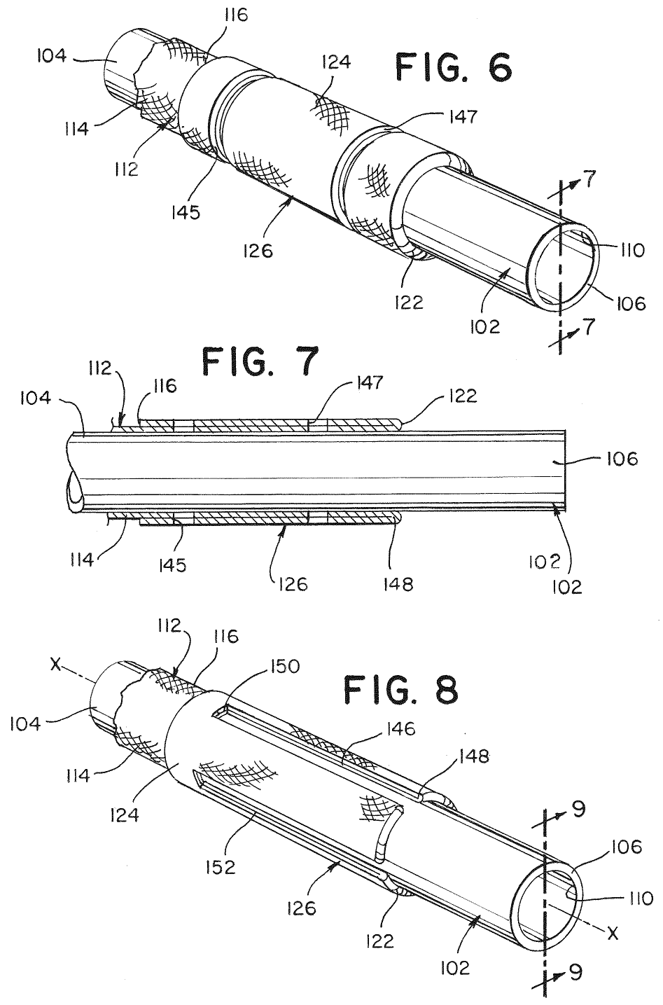

As shown in FIGS. 6 and 7, the cuts 145, 147 in the distal segment 126 can go through both the inner layer and the outer layer of the double braid 124. As shown, the cuts 145, 147 in braid 112 can have an annular shape. While not shown, one skilled in the art will readily appreciate that the cuts 145, 147 can also by disposed at an angle α with respect longitudinal axis X, wherein the angle α is an acute angle.

Referring now to FIGS. 8-10, the distal segment 126 of double braid 124 has a radially inner layer and the radial outer layer of the double braid 124 having a first axial cut or slot 146, wherein the axial cut 146 in the braid extends along the longitudinal axis X. First axial cut 146 in braid 122 has a first end 148 and a second end 150. First end 148 is disposed at the first fold line 122. Second end 150 is disposed within the double braid 124 at the distal segment 126 of the braid proximal from the first end 148. The distal segment 126 of double braid 124 has the radially inner layer and the radial outer layer of the double braid 124 can have a second axial cut 152. Second axial cut or slot 152 in the braid also extends along the longitudinal axis X. Second axial cut 152 in braid 122 has a first end and a second end. The first end of the second axial cut 152 is disposed at the first fold line 122. The second end of the second axial cut 152 is disposed within the double braid 124 at the distal segment 126 of the braid proximal from the first end. The distal segment 126 of double braid 124 has the radially inner layer and the radial outer layer of the double braid 124 having a third axial cut or slot 154. Third axial cut 154 in the braid also extends along the longitudinal axis X. Third axial cut 154 in braid 122 has a first end and a second end. The first end of the third axial cut 154 is disposed at the first fold line 122. The second end of the third axial cut 154 is disposed within the double braid 124 at the distal segment 126 of the braid proximal from the first end. The axial cuts 146, 152, 154 provide a different degree of flexibility due to the different angle of the slots formed by the cuts, while still being reinforced and visible under fluoroscopy.

Referring now to FIG. 11, the double braid 124 has a first end 156 at the first fold line 122 and a second end 158 spaced proximally from the first end 156. The outer layer portion of the double braid 124 has a first tubular portion 160 and a second helical portion 162 that is wrapped about a portion of the braid extending in the helical direction. The first tubular portion 160 is distal from the second helical portion 162. This embodiment could also include cuts 145, 147 in the first tubular section as shown and describe above in FIGS. 6 and 7, or the cuts 146, 152, 154 as shown and describe above in FIGS. 8-10, while the first tubular portion 160 is still being reinforced and visible under fluoroscopy.

Aspects of the invention are also provided by the following numbered clauses:
Clause 1. A catheter 100 for navigating within the vasculature of a body, the catheter comprising:
   a liner 102 having a proximal end 104, a distal end 106, an outer surface 108, an internal lumen 110 extending from the proximal end 104 to the distal end 106, and a longitudinal axis X;
   a braid 112 disposed on the outer surface 108 of the liner 102, the braid 112 having a proximal end 114 and a distal end 116, the braid extending from the proximal end 104 of the liner 102 to the distal end 106 of the liner 102, the braid 112 including a plurality of wires 118, 120; wherein the distal end 116 of the braid is folded over upon itself by a predetermined distance L to form a first fold line 122 and a double braid 124 comprising of a radially inner braid layer and a radially outer layer at a distal segment 126 of the braid 112; and
   a jacket 128 disposed about the braid 112 and extending from the proximal end 104 of the liner 102 to the distal end 106 of the liner 102.
Clause 2. The catheter according to clause 1, wherein a first portion of the wires 118 are made from a radiopaque material and a second portion of the wires 120 are made from a non-radiopaque material.
Clause 3. The catheter according to any one of clauses 1-2, wherein the braid distal end 116 is folded over upon itself to form a second fold line 132 and a first triple braid segment 134 for a portion of the distal segment of the braid and a double braid segment 136 for the remaining portion of the distal segment 126.
Clause 4. The catheter according to any one of clauses 1-3, wherein the first fold line 122 is distal from the second fold line 132.
Clause 5. The catheter according to any one of clauses 1-4, wherein the distal segment 126 of double braid 124 has the radially outer layer of double braid 124 being folded to create a first four-layer braid segment 138 between the distal end 116 of the braid 112 and the second fold line 132.
Clause 6. The catheter according to any one of clauses 1-5, wherein the distal segment 126 of double braid 124 has the radially outer layer of double braid 124 being folded to create a second four-layer braid segment 140 between the distal end 116 of the braid 112 and the second fold line 132.
Clause 7. The catheter according to any one of clauses 1-6, wherein the first four-layer braid segment 138 is distal from and spaced from the second four-layer braid segment 140.
Clause 8. The catheter according to clause 1, wherein the distal segment 126 of double braid 124 has the radially inner layer of the double braid 124 having a first cut 142.
Clause 9. The catheter according to any one of clauses 1-8, wherein the first cut 142 in the braid 112 is in the radially inner portion of the distal segment 126 of the braid such that the folded over portion of the distal segment of the braid is disposed radially over the first cut 126 in the braid 112.
Clause 10. The catheter according to any one of clauses 1-9, wherein the first cut 142 in the braid 112 has an annular shape.
Clause 11. The catheter according to any one of clauses 1-10, wherein the first cut 142 in the braid 112 is at an angle α with respect to the longitudinal axis X, wherein the angle α is an acute angle.
Clause 12. The catheter according to any one of clauses 1-9, wherein the distal segment 126 of double braid 124 has the radially inner layer of the double braid 124 having a second cut 144.
Clause 13. The catheter according to any one of clauses 1-12, wherein the second cut 144 in the braid 112 is in the radially inner portion of the distal segment 126 of the braid such that the folded over portion of the distal segment 126 of the braid is disposed radially over the second cut 144 in the braid.
Clause 14. The catheter according to any one of clauses 1-13, wherein the second cut 144 in the braid 112 has an annular shape.
Clause 15. The catheter according to any one of clauses 1-14, wherein the second cut 144 in the braid 112 is at an angle α with respect to the longitudinal axis X, wherein the angle α is an acute angle.
Clause 16. The catheter according to any one of clauses 1-15, wherein the first cut 142 in the braid 112 is axially spaced from the second cut 144 in the braid 112.
Clause 17. The catheter according to any one of clauses 1-16, wherein the distal segment 126 of double braid 124 has the radially inner layer and the radial outer layer of the double braid 124 having a first axial cut 146, wherein the axial cut 146 in the braid extends along the longitudinal axis X.
Clause 18. The catheter according to any one of clauses 1-17, wherein the first axial cut 146 in the braid 122 has a first end 148 and a second end 150, the first end 148 being disposed at the first fold line 122 and the second end 150 being disposed within the double braid 124 at the distal segment 126 of the braid.
Clause 19. The catheter according to any one of clauses 1-18, wherein the distal segment 126 of double braid 124 has the radially inner layer and the radial outer layer of the double braid 124 having a second axial cut 152, wherein the second axial cut 152 in the braid extends along the longitudinal axis X.
Clause 20. The catheter according to any one of clauses 1-19, wherein the second axial cut 152 in the braid 122 has a first end and a second end, the first end of the second axial cut 152 being disposed at the first fold line 122 and the second end of the second axial cut 152 being disposed within the double braid 124 at the distal segment 126 of the braid.
Clause 21 The catheter according to any one of clauses 1-20, wherein the distal segment 126 of double braid 124 has the radially inner layer and the radial outer layer of the double braid 124 having a third axial cut 154, wherein the third axial cut 154 in the braid extends along the longitudinal axis X.
Clause 22. The catheter according to any one of clauses 1-19, wherein the third axial cut 154 in the braid 122 has a first end and a second end, the first end of the third axial cut 154 being disposed at the first fold line 122 and the second end of the third axial cut 154 being disposed within the double braid 124 at the distal segment 126 of the braid.
Clause 23. The catheter according to any one of clauses 1-22, wherein the double braid 124 has a first end 156 at the first fold line 122 and a second end 158 spaced proximally from the first end 156.
Clause 24. The catheter according to any one of clauses 1-23, wherein the double braid 124 has a first tubular portion 160 and a second helical portion 162 that is wrapped about a portion of the braid.
Clause 25. The catheter according to any one of clauses 1-24, wherein the first tubular portion 160 is distal from the second helical portion 162.

The descriptions contained herein are examples of embodiments of the disclosure and are not intended in any way to limit the scope of the disclosure. As described herein, the disclosure contemplates many variations and modifications of a catheter. Modifications and variations apparent to those having skill in the pertinent art according to the teachings of this disclosure are intended to be within the scope of the claims which follow.

What is claimed is:

1. A catheter for navigating within the vasculature of a body, the catheter comprising:
a liner having a proximal end, a distal end, an outer surface, an internal lumen extending from the proximal end to the distal end, and a longitudinal axis;
a braid disposed on the outer surface of the liner, the braid having a proximal end and a distal end, the braid extending from the proximal end of the liner to the distal end of the liner, the braid including a plurality of wires; wherein the distal end of the braid is folded over upon itself by a predetermined distance to form a first fold line and a double braid comprising of a radially inner braid layer and a radially outer layer at a distal segment of the braid; and
a jacket disposed about the braid and extending from the proximal end of the liner to the distal end of the liner.

2. The catheter according to claim 1, wherein a first portion of the wires are made from a radiopaque material and a second portion of the wires are made from a non-radiopaque material.

3. The catheter according to claim 1, wherein the braid distal end is folded over upon itself to form a second fold line and a first triple braid segment for a portion of the distal segment of the braid and a double braid segment for the remaining portion of the distal segment.

4. The catheter according to claim 3, wherein the first fold line is distal from the second fold line.

5. The catheter according to claim 1, wherein the distal segment of double braid has the radially outer layer of double braid being folded to create a first four-layer braid segment between the distal end of the braid and the second fold line.

6. The catheter according to claim 5, wherein the distal segment of double braid has the radially outer layer of double braid being folded to create a second four-layer braid segment between the distal end of the braid and the second fold line.

7. The catheter according to claim 6, wherein the first four-layer braid segment is distal from and spaced from the second four-layer braid segment.

8. The catheter according to claim 1, wherein the distal segment of double braid has the radially inner layer of the double braid having a first cut.

9. The catheter according to claim 8, wherein the first cut in the braid is in the radially inner portion of the distal segment of the braid such that the folded over portion of the distal segment of the braid is disposed radially over the first cut in the braid.

10. The catheter according to claim 9, wherein the first cut in the braid has an annular shape.

11. The catheter according to claim 10, wherein the first cut in the braid is at an angle with respect to the longitudinal axis, wherein the angle is an acute angle.

12. The catheter according to claim 9, wherein the distal segment of double braid has the radially inner layer of the double braid having a second cut.

13. The catheter according to claim 12, wherein the second cut in the braid is in the radially inner portion of the distal segment of the braid such that the folded over portion of the distal segment of the braid is disposed radially over the second cut in the braid.

14. The catheter according to claim 13, wherein the second cut in the braid has an annular shape.

15. The catheter according to claim 14, wherein the second cut in the braid is at an angle α with respect to the longitudinal axis, wherein the angle α is an acute angle.

16. The catheter according to claim 15, wherein the first cut in the braid is axially spaced from the second cut in the braid.

17. The catheter according to claim 1, wherein the distal segment of double braid has the radially inner layer and the radial outer layer of the double braid having a first axial cut, wherein the axial cut in the braid extends along the longitudinal axis.

18. The catheter according to claim 17, wherein the first axial cut in the braid has a first end and a second end, the first end being disposed at the first fold line and the second end being disposed within the double braid at the distal segment of the braid.

19. The catheter according to claim 18, wherein the distal segment of double braid has the radially inner layer and the radial outer layer of the double braid having a second axial cut, wherein the second axial cut in the braid extends along the longitudinal axis.

20. The catheter according to claim 19, wherein the second axial cut in the braid has a first end and a second end, the first end of the second axial cut being disposed at the first fold line and the second end of the second axial cut being disposed within the double braid at the distal segment of the braid.
